(19) United States
(12) Patent Application Publication
(10) Pub. No.: US 2026/0216390 A1
(43) Pub. Date: Jul. 30, 2026

(54) SYSTEM AND METHOD FOR DECONTAMINATION

(71) Applicant: American Sterilizer Company, Mentor, OH (US)

(72) Inventors: Chris Hayden, Thompson, OH (US); Anthony J. Fiorello, Mentor, OH (US)

(21) Appl. No.: 19/454,255

(22) Filed: Jan. 20, 2026

Related U.S. Application Data
(60) Provisional application No. 63/749,402, filed on Jan. 24, 2025.

(51) Int. Cl. A61L 2/20; A61L 2/084; A61L 2/085; A61L 2/24; A61L 101/34; A61L 101/36; A61L 103/15

(52) U.S. Cl. CPC A61L 2/20; A61L 2/084; A61L 2/085; A61L 2/24; A61L 2101/34; A61L 2101/36; A61L 2103/15; A61L 2202/11; A61L 2202/121; A61L 2202/122; A61L 2202/14; A61L 2202/15

(57) ABSTRACT

Various embodiments disclosed relate to a decontamination method and system. The present disclosure includes a method of sterilizing including applying a vaporized sterilant to a medical instrument or a surface in a sterilization chamber for one or more vapor sterilization cycles, applying blue light to the medical instrument or the surface during the one or more vapor sterilization cycles, and applying near-infrared light to the medical instrument or the surface during the one or more vapor sterilization cycles, simultaneously with the application of the blue light.

### CROSS-REFERENCE TO RELATED APPLICATION

This patent application claims the benefit of U.S. Provisional Patent Application No. 63/749,402, filed Jan. 24, 2025, which is incorporated by reference herein in its entirety.

### BACKGROUND

Sterilization, cleaning, and disinfection of a medical instrument or a surface is an important process. For example, critical surfaces, such as medical devices and cleanrooms, are sterilized prior to use, and/or after use, through a variety of methods and systems.

Vaporized hydrogen peroxide (VHP) sterilization can be used to sterilize such surfaces. VHP is a non-toxic, low-temperature method of sterilization that uses hydrogen peroxide vapor to sterilize medical devices, rooms, and other items. With VHP, a liquid hydrogen peroxide (H₂O₂) solution is vaporized and fills a chamber, where it comes into contact with surfaces and penetrates into deep crevices and hollow lumens. After VHP sterilization, the vapor can be broken down into water and oxygen. VHP can be used, for example, to sterilize critical surfaces for medical devices, e.g., endoscopes, and cleanrooms where pharmaceutical medicines are prepared. VHP is non-toxic, does not require additional ventilation, and is compatible with a variety of materials.

### SUMMARY OF THE DISCLOSURE

In some aspects, the techniques described herein relate to a method of sterilizing a medical instrument or a surface, the method including: applying a vaporized sterilant to the medical instrument or the surface in an enclosed room or chamber for one or more vapor sterilization cycles; applying blue light to the medical instrument or the surface during the one or more vapor sterilization cycles; and applying near-infrared light to the medical instrument or the surface during the one or more vapor sterilization cycles, simultaneously with the application of the blue light.

In some aspects, the techniques described herein relate to a method of sterilizing a medical instrument or a surface, the method including: emitting light in a wavelength range of 400 to 410 nm to the medical instrument or the surface while simultaneously emitting light in a wavelength range of 780 nm to 1060 nm to the medical instrument or the surface for a first time; subsequently treating the medical instrument or the surface with vaporized hydrogen peroxide; and emitting light in a wavelength range of 400 to 410 nm to the medical instrument or the surface while simultaneously applying radiation in a wavelength range of 780 nm to 1060 nm to the medical instrument or the surface for a second time.

In some aspects, the techniques described herein relate to a sterilization or decontamination system including: a sterilization chamber for receipt of one or more medical instrument or one or more objects having one or more surfaces; a hydrogen peroxide source fluidly coupled to the sterilization chamber, wherein the system is configured to vaporize and deliver hydrogen peroxide into the sterilization chamber; a blue light source actuatable to deliver blue light within the sterilization chamber; and a near-infrared light source actuatable to deliver near-infrared light within the sterilization chamber.

In some aspects, the techniques described herein relate to a sterilization or decontamination system including: a room having one or more critical surfaces; a hydrogen peroxide source fluidly coupled to the room, wherein the system is configured to vaporize and deliver hydrogen peroxide into the room; a blue light source actuatable to deliver blue light within the room; and a near-infrared light source actuatable to deliver near-infrared light within the room.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates a vapor hydrogen peroxide system with integrated blue light and near-infrared light in an example.

FIG. 2 illustrates an example method of decontamination.

FIG. 3 illustrates an example method of decontamination.

FIG. 4 illustrates a block diagram of an example computing machine, according to an example.

### DETAILED DESCRIPTION

The present disclosure describes, among other things, a decontamination method using a combination of anti-microbial blue light, near-infrared (NIR) light, and vaporized hydrogen peroxide (VHP) together for synergistic benefits of antimicrobial efficacy. The method can include use of blue light (e.g., in a 400 to 410 nm range) and NIR light (e.g., in a 780 to 1060 nm wavelength range) in combination before and/or during a VHP decontamination process to more effectively decontaminate one or more medical instruments or surfaces. For example, both types of lights can be left on throughout the VHP process to boost antimicrobial efficacy, such as throughout the VHP injection and vaporization steps. These methods can be applied to existing VHP processes.

There can be a synergistic relationship between NIR light, anti-microbial blue light (e.g., 405 nm), and oxidative processes. Adding these elements together in a VHP process can exhibit an advantage in efficacy and efficiency compared to other VHP decontamination processes.

This combination can help improve VHP technology without affecting material compatibility or adding additional safety processions. In an example, this can help speed up the liberation and destruction of VHP molecules from VHP treated surfaces, also known as aeration, by breaking down and heating up hydrogen peroxide and peracetic acid molecules upon prolonged exposure to the NIR and blue light. The combination allows for reduced VHP such that decontamination may be done safely in open spaces without the need for personal protective equipment or special isolation. For example, NIR light is already used in various applications like security cameras and has no skin exposure concerns, while 405 nm blue light is considered safe for continuous skin exposure for up to 24 hours while providing antimicrobial effects. In some examples, using combined 405 nm blue light and NIR light can create a 4-log reduction and its own to surfaces the light can touch. These specific light ranges also show a synergistic effect with oxidative chemistries.

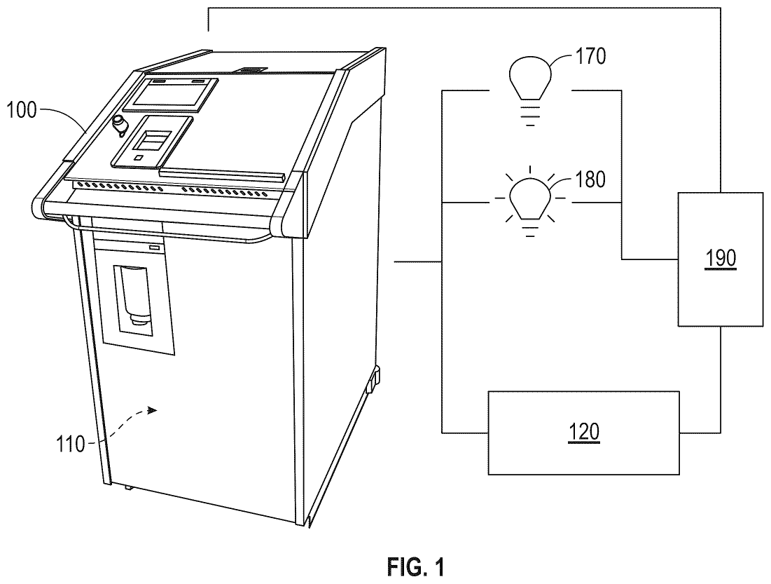

FIG. 1 illustrates a vapor hydrogen peroxide system 100 with integrated blue light and near-infrared light in an example. The system 100 can include a chamber 110, a hydrogen peroxide source 120, the blue light source 170, the near-infrared light source 180, and a controller 190.

The system 100 can be a vapor hydrogen peroxide system that allows for sterilization of items, such as instruments and devices, within the sterilization chamber 110, by way of vaporized hydrogen peroxide. In some cases, the vapor hydrogen peroxide system 100 can be a stationary system with an internal chamber. In some cases, the vapor hydrogen peroxide system 100 can be a mobile or portable system. In some cases, the vapor hydrogen peroxide system 100 can be used to sterilize an environment, such as a clean room or other location.

The vapor hydrogen peroxide system 100 can be used for pharmaceutical grade biological decontamination for isolators, small enclosure devices, and cleanrooms.

In the example of FIG. 1, the sterilization chamber 110 can be sized and shaped for receipt of one or more medical instruments or devices, such as lumened instruments like endoscopes. The sterilization chamber 110 can be sealable from an external environment, and be made of material suitable for containment of vaporized hydrogen peroxide. The sterilization chamber 110 can be fluidly connected to other components by use of tubing or other channels made of similarly suitable materials not corrodible by hydrogen peroxide.

The hydrogen peroxide source 120 can be fluidly coupled to the sterilization chamber 110. Hydrogen peroxide (H₂O₂) can be stored in the source 120, which can be, for example a tank or other suitable container. Hydrogen peroxide can be pumped from the hydrogen peroxide source 120 to the sterilization chamber 110, where it can be vaporized. The vaporized hydrogen peroxide can fill the sterilization chamber 110 and flow in, on, and around, any medical instruments or surface(s) or device(s) therein, including through channels or lumens of those instruments, so as to sterilize the items.

The blue light source 170 can be actuatable to deliver blue light within the room or sterilization chamber 110. In an example, the blue light source 170 can be integrated with the room or sterilization chamber 110. In an example, the blue light source 170 can be separable from the room or sterilization chamber 110. In some cases, the blue light source 170 can be fixed within the room or sterilization chamber 110 and electrically connected via one or more wires to a power source external to the room or sterilization chamber 110. In some cases, the blue light source 170 can be battery powered. The blue light source 170 can optionally be cased, housed, or covered, as needed to withstand corrosion from hydrogen peroxide vapor. In some cases, the blue light source 170 can be external to the room or sterilization chamber 110, but aligned such that it shines blue light therein, such as through a window, door, or other non-blue light blocking component.

The blue light source 170 can be a diode, laser, or other light source configured to emit blue light throughout the room or sterilization chamber 110 such that any objects, such as medical instruments or critical surfaces or devices therein, are bathed in blue light when the blue light source 170 is continuously emitting or intermittently pulsed to activate and apply inactivating energy to the VHP treated surface being decontaminated.

In an example, the blue light source 170 can emit light in the wavelength range of about 380 to 500 nm, about 390 to 490 nm, 400 to 480 nm, 410 to 470 nm, 410 to 460 nm, 420 to 450 nm, or 430 to 440 nm. For example, the wavelength of the blue light can be about 400 nm, about 405 nm, or about 410 nm.

In an example, anti-microbial blue light at 405 nm can be used in industry and is safe for 24 hours of continuous skin exposure. Blue light at 405 nm can provide a 2-log reduction of bacterial contamination over thirty minutes. Similarly, blue light outside the ultraviolet (UV) range, such as at 400 nm or below, can have such positive benefits.

Overall, blue light (e.g., 405 nm), can have significant antimicrobial properties against a wide variety of bacterial and fungal pathogens, while still being safe for facility use. The blue light can interact with hydrogen peroxide to synergistically increase anti-microbial effects through the production of germicidal free radicals.

The near-infrared light source 180 can be actuatable to deliver infrared light within the room or sterilization chamber 110. In some cases, the near-infrared light source 180 can be integrated with the room or sterilization chamber 110. In some cases, the near-infrared light source 180 can be separable from the sterilization chamber 110. In some cases, the near-infrared light source 180 can be fixed within the room or sterilization chamber 110 and electrically connected via one or more wires to a power source external to the room or sterilization chamber 110. In some cases, the near-infrared light source 180 can be battery powered. The near-infrared light source 180 can optionally be cased, housed, or covered, as needed to withstand corrosion from hydrogen peroxide vapor. In some cases, the near-infrared light source 180 can be external to the room or sterilization chamber 110, but aligned such that it shines red light therein, such as through a window, door, or other non-IR light blocking component.

In an example, the near-infrared light source 180 can emit near-infrared or infrared light wavelengths, such as in a range of about 750 to 2,500 nm, 800 to 2,450 nm, 850 to 2,400 nm, 900 to 2,350 nm, 950 to 2,300 nm, 1,000 to 2,250 nm, 1,050 to 2,200 nm, 1,100 to 2,150 nm, 1,150 to 2,150 nm, 1,200 to 2,100 nm, 1,250 to 2,050 nm, 1,300 to 2,000 nm, 1,350 to 1,950 nm, 1,400 to 1,900 nm, 1,450 to 1,850 nm, 1,500 to 1,800 nm, 1,550 to 1,750 nm, or 1,600 to 1,700 nm.

Near-IR light can provide a 0.5 log reduction alone, and can also synergistically work to accelerate other reactions. Near-IR wavelengths do not have skin exposure concerns, and near-IR can boost the anti-microbial activity of hydrogen peroxide.

Near-IR light can induce physiological effects through two types of photo acceptors: cytochrome c oxidase and intracellular water. Photo absorption can convert light into signals that stimulate biological processes. In an example, energy in the photons from IR and blue light can damage microorganism's cellular membranes, DNA, and proteins by breaking atomic and molecular bonds. The action of IR light on water dynamics in membranes, mitochondria and/or cells could modulate signaling pathways, and produce reactive oxygen species (ROS), ATP (adenosine triphosphate), Calcium ions (Ca²⁺), nitrous oxide (NO), and inositol phosphates groups. Secondary effects of IR light can include stress signaling, metabolic processes, cytoskeleton organization, cell proliferation/differentiation, and homeostasis. These effects can result in anti-microbial action when NIR light is applied, such as in the sterilization chamber 110.

In another example, IR light can excite cells through water absorption, with a temperature increase affecting the plasma membrane and altering the electrical capacitance, thereby depolarizing the target cells. In this case, photon absorption can result in a rapid increase in intracellular temperature which may promote physiological changes in temperature, pH, osmosis, and ATP yield, potentially resulting in an anti-microbial effect.

Overall, IR application, including near-IR application, can create radiation-induced antimicrobial properties effective for bacteria reduction, such as against Escherichia coli, Staphylococcus aureus, and Klebsiella pneumoniae, among other species. IR can include light in the wavelength range of about 750 nm to 1 mm, while near-IR light is a subset of IR light that can include light in the wavelength range of about 750 nm to 2500 nm.

In another example, an IR humidifier can be used as the near-infrared light source 180. In an example, IR light could be used for dehumidification of the decontamination space to allow more VHP to fill the area without condensing. For example, an IR humidifier can be installed using a high intensity quartz IR lamp and a corresponding humidifier pan. Here, the IR radiation from the lamp can break the surface tension on water in the pan, allowing air flow across the pan to evaporate and carry the moisture away as vapor, providing both IR light and humidification. This can allow for improved distribution and size of vaporized molecules resulting in less heat of vaporization and less condensation formation.

In some cases, the blue light source 170 and the near-infrared light source 180 can be integrated with each other, such as having a single bulb, lamp, diode, or laser; or multiple light sources, such as bulbs, lamps, diodes, or laser, combined in a single light-emitting device.

A synergistic effect can be seen between near-IR (e.g., 780 nm~1060 nm), blue light (e.g., 380 nm~500 nm), and hydrogen peroxide vapor. Near-IR can allow for cytochrome oxidase binding with oxygen to turn on protectors and stimulate cell metabolism. Blue light, on the other hand, can cause a toxic environment when the immune response has been triggered. Using both in combinations with hydrogen peroxide can allow for boosted anti-microbial activity, and potentially reduce cycle times required for decontamination.

The controller 190 can allow for control of the vapor hydrogen peroxide system 100, including the blue light source 170 and the near-infrared light source 180, and control of the release and vaporization of hydrogen peroxide. The controller 190 can be in communication with the sterilization chamber 110, the hydrogen peroxide source 120, the blue light source 170, and the near-infrared light source 180. The controller 190 can be manually actuated, partially automated, or fully automated. In some cases, the controller 190 can initiate or regulate a process cycle within the vapor hydrogen peroxide system 100.

For example, the controller 190 can initiate the closure of the sterilization chamber 110, release of hydrogen peroxide therein, and/or the application of blue light and/or near-IR light in the sterilization chamber 110 to sterilize the items in the sterilization chamber 110. The application of hydrogen peroxide, blue light, and/or near-IR light can be performed according to a pattern or schedule, or in a manual manner, or in an automated or semi-automated manner, such as over the course of more than one cycle of the vapor hydrogen peroxide system 100.

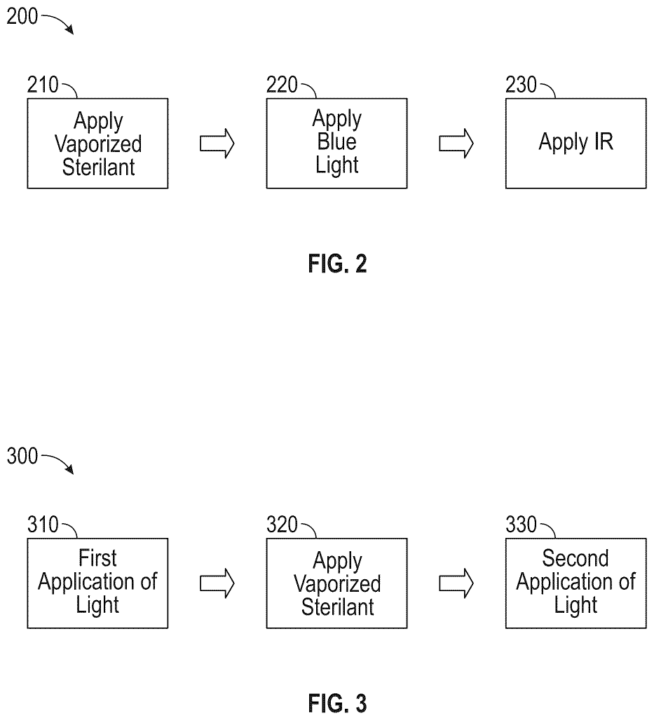

FIGS. 2 and 3 depict various methods of decontamination, such as using the example system 100 discussed above. The methods of FIGS. 2 and 3 leverage a combination of blue light, near-IR light, and hydrogen peroxide vapor.

FIG. 2 illustrates an example method 200 of decontamination. The method can include blocks 210 to 230. The method 200 can, for example, be used to sterilize a surface or device.

At block 210, the method can include applying a vaporized sterilant to the instrument or surface in a sterilization chamber. The vaporized sterilant can be, for example, hydrogen peroxide. In an example, a single or multiple instruments or devices can be in the sterilization chamber. In some cases, other types of medical products can be treated in method 200. In an example, the application of a vaporized sterilant can be performed in single or multiple cycles, in a manual, automated, or semi-automated fashion, such as directed by a controller or computing device. In an example, the sterilant can be hydrogen peroxide or peracetic acid. In an example, the sterilization chamber can be part of a vaporized hydrogen peroxide processing machine. Applying the vaporized sterilant can include vaporizing the sterilant in the chamber for a predetermined process cycle time.

At block 220, blue light can be applied to the instrument in the sterilization chamber. At block 230, near-infrared (IR) light can be applied to the instrument in the sterilization chamber. In an example, blue light and near-IR light can be applied simultaneously. The light can be applied before, during, or after the vaporization of the sterilant. The light(s) can be applied simultaneously, in sequence, in a pattern, continuously, or intermittently. The light(s) can be applied via individual light sources, combined light sources, and/or one or more light sources integrated with a sterilization system. For example, diodes, lasers, bulbs, lamps, or other types of light sources may be used. The blue light can be provided, for example, in a wavelength range of about 400 nm to 410 nm. The near-IR light can be provided, for example, in a wavelength range of 780 nm to 1060 nm.

In some cases, the blue light, the near-IR light, or both, can be applied at the beginning or prior to a vapor sterilization cycle. In some cases, applying blue light and simultaneously applying near-infrared light can be done continuously throughout at least one of the sterilization cycles. In some cases, applying blue light and simultaneously applying near-infrared light can be performed according to a predetermined pattern during the sterilization cycle. In some cases, applying blue light and near-IR light to the medical instrument or critical surface can be performed after the one or more sterilization cycles have concluded.

FIG. 3 illustrates an example method 300 of decontamination, such as a method of sterilizing a medical instrument or a critical surface. The method 300 can include block 310 to 330.

At block 310, for a first time, light can be emitted to the medical instrument or critical surface in a wavelength range of 780 nm to 1060 nm. Simultaneously, light can be emitted to the medical instrument or a critical surface in a wavelength range of 780 nm to 1060 nm.

At block 320, the medical instrument or critical surface can subsequently be treated with vaporized hydrogen peroxide. In some cases, the medical instrument or critical surface can be repeatedly treated.

At block 330, for a second time, light can be emitted to the medical instrument or the critical surface in a wavelength range of 780 nm to 1060 nm. Simultaneously, light can be emitted to the medical instrument or the critical surface in a wavelength range of 780 nm to 1060 nm.

In some cases, the medical instrument or critical surface can be further treated by applying radiation in a range of 400 to 410 nm to the medical instrument or the critical surface and applying radiation in a range of 780 nm to 1060 nm to the medical instrument or the critical surface for a third time after treating the medical instrument or the critical surface with vaporized hydrogen peroxide.

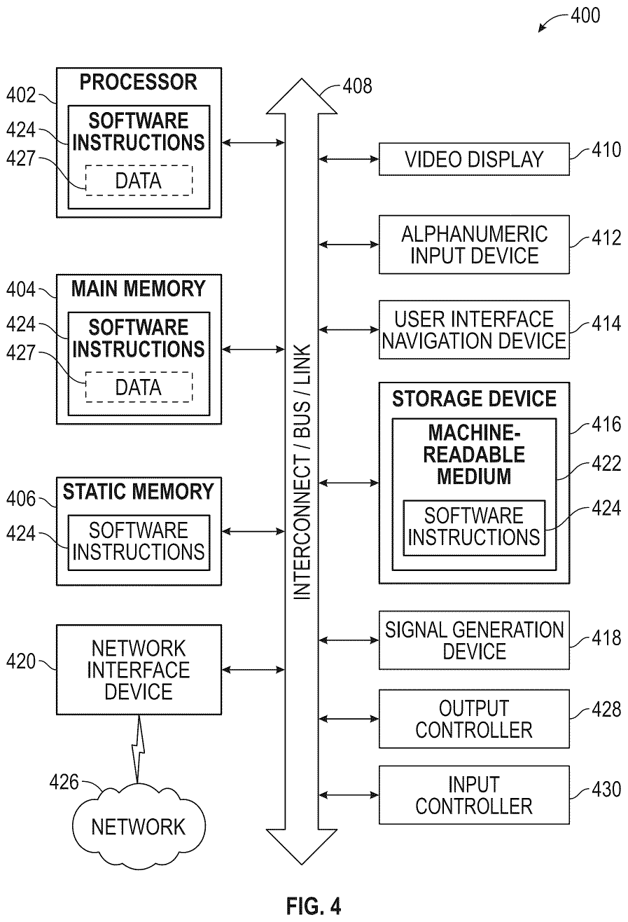

FIG. 4 illustrates a block diagram of an example machine 400 (e.g., computer system, computing device, machine, controller, etc.) that may be programmed into a special purpose machine suitable for implementing one or more embodiments for data processing, data communication, user interface, or like aspects disclosed herein. For instance, the system 100 and its controller 190 described above may be embodied by the machine 400, such as in the form of a computer or specialized electronic device that includes sufficient processing power, memory resources, and communications throughput capability to perform specific compute operations consistent with the examples herein.

The machine 400 may include a hardware processor 402 (e.g., a microprocessor, a central processing unit (CPU), a graphics processing unit (GPU), a hardware processor core, or any combination thereof), a main memory 404 and a static memory 406, some or all of which may communicate with each other via an interconnect, link or bus 408. The machine 400 may further include a display unit 410, an alphanumeric input device 412 and a user interface (UI) navigation device 414. In an example, the display unit 410, alphanumeric input device 412 and navigation device 414 may be a touch screen display. The machine 400 may additionally include a storage device 416 (e.g., drive unit), a signal generation device 418 (e.g., an audio or radio signal generation device), and a network interface device 420 (e.g., for connectivity with a network). The machine 400 may include an output controller 428, such as a serial (e.g., universal serial bus (USB), parallel, or other wired or wireless (e.g., Bluetooth, near field communication (NFC), etc.) connection to communicate or control one or more peripheral devices, and an input controller 430 to connect to more sensors.

The storage device 416 may include a machine readable medium 422 that is non-transitory on which is stored one or more sets of data structures or instructions 424 (e.g., software) embodying or utilized by any one or more of the techniques or functions described herein. The instructions 424 may also reside, completely or at least partially, within the main memory 404, within static memory 406, or within the hardware processor 402 during execution thereof by the machine 400. In an example, one or any combination of the hardware processor 402, the main memory 404, the static memory 406, or the storage device 416 may constitute machine readable media.

The term “machine readable medium” may include any medium that is capable of storing, encoding, or carrying instructions for execution by the machine 400 and that cause the machine 400 to perform any one or more of the techniques of the present disclosure, or that is capable of storing, encoding or carrying data structures 427 used by or associated with such instructions. Non-limiting machine-readable medium examples may include solid-state memories, and optical and magnetic media. Specific examples of machine-readable media may include non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; and optical storage disks.

The instructions 424 may further be transmitted or received over a communications network 426 using a transmission medium via the network interface device 420 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), and wireless data networks (e.g., Institute of Electrical and Electronics Engineers (IEEE) 802.11 family of standards known as Wi-Fi®), IEEE 802.15.4 family of standards, peer-to-peer (P2P) networks, among others. In an example, the network interface device 420 may include one or more physical jacks or one or more antennas to connect to the communications network 426. In an example, the network interface device 420 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding or carrying instructions for execution by the machine 400, and includes digital or analog communications signals or other intangible medium to facilitate communication of such software.

The devices described herein may be configured to include computer-readable non-transitory media storing computer readable instructions and one or more processors coupled to the memory, and when executing the computer readable instructions configure the machine 400 to perform steps and operations described above for electronic systems or devices (e.g., to display a user interface and receive user interface commands, perform sensing operations from electromechanical and environmental sensors, extract and identify data values, etc.). The computer-readable non-transitory media includes all types of computer readable media, including magnetic storage media, optical storage media, flash media and solid-state storage media. It should be further understood that software including one or more computer-executable instructions that facilitate processing and operations as described above with reference to any one or all of steps of the disclosure may be installed in and sold with networked devices (e.g., servers or cloud computing systems) consistent with the disclosure. Alternatively, the software may be obtained and loaded (or, re-loaded/upgraded) from one or more servers and/or cloud computing systems, such as software stored on a server for distribution over the Internet, for example.

Method examples or other operations described herein can be machine or device (e.g., computer, robotic) implemented at least in part. The components of the illustrative devices, systems and methods employed in accordance with the illustrated embodiments may be implemented, at least in part, in digital electronic circuitry, analog electronic circuitry, or in computer hardware, firmware, software, or in combinations of them. These components may be implemented, for example, as a computing program product such as a computing program, program code or computer instructions tangibly embodied in an information carrier, or in a machine-readable storage device, for execution by, or to control the operation of, a data processing apparatus such as a programmable processor, a computer, or multiple computers. A computing program may be written in any form of programming language, including compiled or interpreted languages, and it may be deployed in any form, including as a stand-alone program or as a module, component, subroutine, or other unit suitable for use in a computing environment. Also, functional programs, codes, and code segments for accomplishing the techniques described herein may be easily construed as within the scope of the present disclosure by programmers skilled in the art. Method steps associated with the illustrative embodiments may be performed by one or more programmable processors executing a computing program, code or instructions to perform functions (e.g., by operating on input data and/or generating an output). Method steps may also be performed by, and apparatus may be implemented as, special purpose logic circuitry, e.g., an FPGA (field programmable gate array) or an ASIC (application-specific integrated circuit), for example.

Thus, in implementation in a controller or other machine for medical item processing, various logical blocks, modules, and circuits described in connection with the embodiments disclosed herein may be implemented or performed with a general-purpose processor, a digital signal processor (DSP), an ASIC, a FPGA or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration. Processors suitable for the execution of a computing program include, by way of example, both general and special purpose microprocessors, and any one or more processors of any kind of digital computer. Information carriers suitable for embodying computing program instructions and data include all forms of non-volatile memory, including by way of example, semiconductor memory devices, e.g., electrically programmable read-only memory or ROM (EPROM), electrically erasable programmable ROM (EEPROM), flash memory devices, and data storage disks (e.g., magnetic disks, internal hard disks, or removable disks, etc.). The processor and the memory may be supplemented by or incorporated in special purpose logic circuitry.

As used herein, “machine-readable medium” or “machine-readable storage medium” means a device able to store instructions and data temporarily or permanently and may include, but is not limited to, random-access memory (RAM), read-only memory (ROM), buffer memory, flash memory, optical media, magnetic media, cache memory, other types of storage (e.g., Erasable Programmable Read-Only Memory (EEPROM)), and/or any suitable combination thereof. The term “machine-readable medium” or “machine-readable storage medium” should be taken to include a single medium or multiple media (e.g., a centralized or distributed database, or associated caches and servers) able to store processor instructions. The term “machine-readable medium” or “machine-readable storage medium” shall also be taken to include any medium, or combination of multiple media, which is capable of storing instructions for execution by one or more processors (or other processing circuitry), such that the instructions, when executed by one or more processors cause the one or more processors to perform any one or more of the methodologies described herein. Accordingly, a “machine-readable medium” or “machine-readable storage medium” refers to a single storage apparatus or device, as well as “cloud-based” storage systems or storage networks that include multiple storage apparatus or devices. A non-transitory “machine-readable medium” or “machine-readable storage medium” as used herein excludes signals per se.

### EXAMPLES

Various embodiments of the present disclosure can be better understood by reference to the following Examples which are offered by way of illustration. The present disclosure is not limited to the Examples given herein.

### Example

### IR and Blue Light Application with H₂O₂

The combination of 405 nm and NIR light after VHP can create a 4-log reduction on surfaces the light can touch. These specific light ranges show a synergistic effect with oxidative chemistries like hydrogen peroxide. In an example, an IR device and a blue light source can be used in conjunction with a vaporized hydrogen peroxide (VHP) system.

The IR device can, for example, be a diode laser light device. For example, the Epic X, Biolase can be used to emit NIR light with a wavelength of about 940 nm (±10 nm) for about 30 seconds at a time. The blue light source can be a light configured to produce 405 nm wavelength light. The IR device and the blue light source can be programmed as desired to apply such light in combination with the hydrogen peroxide.

The terms and expressions that have been employed are used as terms of description and not of limitation, and there is no intention in the use of such terms and expressions of excluding any equivalents of the features shown and described or portions thereof, but it is recognized that various modifications are possible within the scope of the embodiments of the present disclosure. Thus, it should be understood that although the present disclosure has been specifically disclosed by specific embodiments and optional features, modification and variation of the concepts herein disclosed may be resorted to by those of ordinary skill in the art, and that such modifications and variations are considered to be within the scope of embodiments of the present disclosure.

### Additional Embodiments

The following exemplary embodiments are provided, the numbering of which is not to be construed as designating levels of importance:

In some aspects, the techniques described herein relate to a method of sterilizing a medical instrument or surface, the method including: applying a vaporized sterilant to the medical instrument or the surface in a sterilization chamber for one or more vapor sterilization cycles; applying blue light to the medical instrument or surface during the one or more vapor sterilization cycles; and applying near-infrared light to the medical instrument or surface during the one or more vapor sterilization cycles, simultaneously with the application of the blue light.

In some aspects, the techniques described herein relate to a method, further including applying blue light, near-infrared light, or both to the medical instrument or surface prior to a beginning of the one or more vapor sterilization cycles.

In some aspects, the techniques described herein relate to a method, wherein the blue light is in a wavelength range of 400 nm to 410 nm.

In some aspects, the techniques described herein relate to a method, wherein the near-infrared light is in a wavelength range of 780 nm to 1060 nm.

In some aspects, the techniques described herein relate to a method, wherein applying blue light and simultaneously applying near-infrared light are performed continuously throughout at least one of the one or more vapor sterilization cycles.

In some aspects, the techniques described herein relate to a method, wherein applying blue light and simultaneously applying near-infrared light are performed according to a predetermined pattern during the one or more vapor sterilization cycles.

In some aspects, the techniques described herein relate to a method, further including simultaneously applying blue light and near-infrared light to the medical instrument or the surface after the one or more sterilization cycles have concluded.

In some aspects, the techniques described herein relate to a method, wherein the sterilant is hydrogen peroxide or peracetic acid.

In some aspects, the techniques described herein relate to a method, wherein the sterilization chamber is part of a vaporized hydrogen peroxide processing machine.

In some aspects, the techniques described herein relate to a method, wherein applying the vaporized sterilant includes vaporizing the sterilant in the chamber for a predetermined process cycle time.

In some aspects, the techniques described herein relate to a method of sterilizing a medical instrument or a surface, the method including: emitting light in a wavelength range of 400 to 410 nm to the medical instrument or surface while simultaneously emitting light in a wavelength range of 780 nm to 1060 nm to the medical instrument or surface for a first time; subsequently treating the medical instrument or surface with vaporized hydrogen peroxide; and emitting light in a wavelength range of 400 to 410 nm to the medical instrument or surface while simultaneously applying radiation in a wavelength range of 780 nm to 1060 nm to the medical instrument or surface for a second time.

In some aspects, the techniques described herein relate to a method, wherein emitting light in a range of 400 to 410 nm to the medical instrument or surface and emitting light in a range of 780 nm to 1060 nm to the medical instrument or surface are performed simultaneously.

In some aspects, the techniques described herein relate to a method, further including repeatedly treating the medical instrument or surface with vaporized hydrogen peroxide.

In some aspects, the techniques described herein relate to a method, further including subsequently further including applying radiation light in a wavelength range of 400 to 410 nm to the medical instrument or surface and applying radiation light in a wavelength range of 780 nm to 1060 nm to the medical instrument or surface for a third time after treating the medical instrument or surface with the vaporized hydrogen peroxide.

In some aspects, the techniques described herein relate to a decontamination or sterilization system including: a sterilization chamber for receipt of a medical instrument or a surface; a hydrogen peroxide source fluidly coupled to the sterilization chamber, wherein the system is configured to vaporize and deliver hydrogen peroxide into the sterilization chamber; a blue light source actuatable to deliver blue light within the sterilization chamber; and a near-infrared light source actuatable to deliver near-infrared light within the sterilization chamber.

In some aspects, the techniques described herein relate to a sterilization system, wherein the blue light source and the near-infrared light source are integrated with each other.

In some aspects, the techniques described herein relate to a sterilization system, further including a controller configurable to control a sterilization cycle on the system.

In some aspects, the techniques described herein relate to a sterilization system, wherein the blue light source is a diode, laser, bulb, or lamp.

In some aspects, the techniques described herein relate to a sterilization system, wherein the near-infrared light source is a diode, laser, bulb, or lamp.

What is claimed is:

1. A method of sterilizing a medical instrument or a surface, the method comprising:
applying a vaporized sterilant to the medical instrument or the surface in a sterilization chamber for one or more vapor sterilization cycles;
applying blue light to the medical instrument or the surface during the one or more vapor sterilization cycles; and
applying near-infrared light to the medical instrument or the surface during the one or more vapor sterilization cycles, simultaneously with the application of the blue light.

2. The method of claim 1, further comprising applying blue light, near-infrared light, or both to the medical instrument or the surface prior to a beginning of the one or more vapor sterilization cycles.

3. The method of claim 1, wherein the blue light is in a wavelength range of 400 nm to 410 nm.

4. The method of claim 1, wherein the near-infrared light is in a wavelength range of 780 nm to 1060 nm.

5. The method of claim 1, wherein applying blue light and simultaneously applying near-infrared light are performed continuously throughout at least one of the one or more vapor sterilization cycles.

6. The method of claim 1, wherein applying blue light and simultaneously applying near-infrared light are performed according to a predetermined pattern during the one or more vapor sterilization cycles.

7. The method of claim 1, further comprising simultaneously applying blue light and near-infrared light to the medical instrument or the surface after the one or more vapor sterilization cycles have concluded.

8. The method of claim 1, wherein the sterilant is hydrogen peroxide or peracetic acid.

9. The method of claim 1, wherein the sterilization chamber is part of a vaporized hydrogen peroxide processing machine.

10. The method of claim 1, wherein applying the vaporized sterilant comprises vaporizing the sterilant in the chamber for a predetermined process cycle time.

11. A method of sterilizing a medical instrument or a surface, the method comprising:
emitting light in a wavelength range of 400 to 410 nm to the medical instrument or the surface while simultaneously emitting light in a wavelength range of 780 nm to 1060 nm to the medical instrument or the surface for a first time;
subsequently treating the medical instrument or the surface with vaporized hydrogen peroxide; and
emitting light in a wavelength range of 400 to 410 nm to the medical instrument or the surface while simultaneously applying radiation in a wavelength range of 780 nm to 1060 nm to the medical instrument or the surface for a second time.

12. The method of claim 11, wherein emitting light in a range of 400 to 410 nm to the medical instrument or the surface and emitting light in a range of 780 nm to 1060 nm to the medical instrument or the surface are performed simultaneously.

13. The method of claim 11, further comprising repeatedly treating the medical instrument or the surface with the vaporized hydrogen peroxide.

14. The method of claim 11, further comprising subsequently further comprising applying radiation light in a wavelength range of 400 to 410 nm to the medical instrument or the surface and applying radiation light in a wavelength range of 780 nm to 1060 nm to the medical instrument or the surface for a third time after treating the medical instrument or the surface with the vaporized hydrogen peroxide.

15. A decontamination system comprising:
a sterilization chamber for receipt of a medical instrument or a surface;
a hydrogen peroxide source fluidly coupled to the sterilization chamber, wherein the system is configured to vaporize and deliver hydrogen peroxide into the sterilization chamber;
a blue light source actuatable to deliver blue light within the sterilization chamber; and
a near-infrared light source actuatable to deliver near-infrared light within the sterilization chamber.

16. The decontamination system of claim 15, wherein the blue light source and the near-infrared light source are integrated with each other.

17. The decontamination system of claim 15, further comprising a controller configurable to control a sterilization cycle of the system.

18. The decontamination system of claim 15, wherein the blue light source is a diode, laser, bulb, or lamp.

19. The decontamination system of claim 15, wherein the near-infrared light source is a diode, laser, bulb, or lamp.
